(19) United States
(12) Patent Application Publication
(10) Pub. No.: US 2005/0042228 A1
(43) Pub. Date: Feb. 24, 2005

(54) Method and pharmaceutical composition for the treatment of immune disorders

(76) Inventor: Che-Chun Su, Taichung (TW)

Correspondence Address: Jackson Walker L.L.P.;Suite 2100, 112 E. Pecan Street, San Antonio, TX, 78205, US

(21) Appl. No.: 10/643,400

(22) Filed: Aug. 19, 2003

(51) Int. Cl. A61K 39/00; C12Q 1/68

(52) U.S. Cl. 424/185.1; 514/12

(57) ABSTRACT

The present invention relates to a method for treating immune disorders and a method for lowering the levels of T-cell activation and proliferation in a human comprising administering CD30 or a biologically functional equivalent thereof to a human afflicted with immune disorders. The present invention also relates to a pharmaceutical composition for treating immune disorders in a human comprising a therapeutically effective amount of CD30 or a biologically functional equivalent thereof, and a pharmaceutically acceptable carrier, excipient or diluent.

### BACKGROUND OF THE INVENTION

1. Field of the Invention

The present invention relates generally to the treatment of immune disorders and more specifically to suppress the levels of T-cell activation and proliferation in immune disorders. More particularly, the present invention provides a pharmaceutical composition and a method of using CD30 or a biologically functional equivalent thereof to suppress the levels of T-cell activation and proliferation in immune disorders.

2. Description of Related Art

Hodgkin's disease (HD) is characterized by numerous infiltrating immune cells, especially T cells, surrounding malignant cells, which comprise less than one percent of the total cell population in the tumor (Cossman et al., Lab. Invest. 78:229, 1998). The tumor cells clearly succeed in evading attacks by these immune cells.

The neoplastic cells (i.e., Hodgkin and Reed-Sternberg, H-RS cells) in HD are characterized by an abundant expression of CD30, a member of the TNFR (tumor necrosis factor receptor) superfamily (Durkop et al., Cell, 68:421, 1992). CD30 is a 120 kd surface antigen widely used as a clinical marker for HD.

CD30 was originally thought to be a receptor that carried out its function through engagement with its respective ligand, CD30L (CD153) (Smith et al. Cell, 73:349, 1993), expressed as a type II membrane glycoprotein in activated T cells, and other types of cells (Nicod et al., Am. J. Resp. Cell. Mol. Biol., 17:91, 1997, Pinto et al., Blood., 88:3299, 1996). However, recent studies have shown a bi-directional instead of uni-directional signal transduction after the CD30-CD30L engagement in T and B cells (Shanebeck et al., Eur. J. Immunol., 25:2147, 1995; Wiley et al., J. Immunol., 157:3635, 1996). CD30L cross-linking has been shown to induce gene expression and metabolic activity in human T cells and neutrophils. Engagement of CD30L by CD30 on T cells inhibits class switching as well as IgG, IgA, and IgE production in B cells (Cerutti et al., Nat. Immunol., 2:150, 2001; Cerutti et al., J. Immunol., 165:786, 2000). A viral counterpart of CD30 has also been shown to function in a similar fashion. (Saraiva et al., J. Exp. Med., 196: 829, 2002.)

The role of CD30-CD30L interaction in health and disease is still not totally understood. Potential therapeutic uses for CD30 or a biologically functional equivalent thereof have yet to be identified.

### SUMMARY OF THE INVENTION

An objective of the present invention is to provide a method for treating immune disorders by lowering the levels of T-cell activation and proliferation by administering CD30 or a biologically functional equivalent thereof to a human afflicted with immune disorders.

The other objective of the present invention is to provide a pharmaceutical composition for treating immune disorders in a human comprising a therapeutically effective amount of CD30 or a biologically functional equivalent thereof, and a pharmaceutically acceptable carrier, excipient or diluent.

Further benefits and advantages of the present invention will become apparent after a careful reading of the detailed description with appropriate reference to the accompanying drawings.

### BRIEF DESCRIPTION OF THE DRAWINGS

FIGS. 1A, 1B and 1C are schematic diagrams showing the construction of a recombinant soluble human CD30-Fc chimeric protein and show the monomeric structure of the recombinant human CD30-Fc fusion protein;

FIG. 2 shows the construction of plasmid pMIB rhu CD30-Fc;

FIGS. 3A and 3B are graphs showing the inhibitory effect of either plate-bound or soluble CD30-Fc fusion protein on T-cell proliferation;

FIG. 4 is a graph showing the inhibitory effect of CD30-Fc fusion protein on the production of IL-2 by anti-CD3-stimulated T cells;

FIG. 5 is a graph showing the effect of IL-2 addition on T-cell proliferation in the presence of CD30-Fc fusion protein; and

FIG. 6 shows the effect of CD30-Fc fusion protein on the inhibition of CD25 and CD26 expression in anti-CD3-stimulated T cells.

### DETAILED DESCRIPTION OF THE INVENTION

Definitions

As used herein, the term “CD30” refers to a protein having an amino acid sequence substantially similar to the amino acid sequence of the native mammalian CD30 protein, which are capable of binding to CD30 ligand molecules. In the absence of any species designation, CD30 refers generically to mammalian CD30. Similarly, in the absence of any specific designation, the term CD30 means all forms of CD30.

The term “biologically functional equivalent thereof,” as used throughout the specification, means that the function of a substance described as “the biologically functional equivalent” is the same as the function of CD30. Preferably, the function of the biologically functional equivalent thereof is capable of binding to CD30 ligand and transmitting a stimulus to a cell. More preferably, the biologically functional equivalent within the scope of the present invention is capable of binding to CD30 ligand and inhibiting the levels of T cell proliferation and/or activation. Preferably, the biologically functional equivalent may be an analog, a subunit, a mutant, an agonist polypeptide, a chimeric protein or a derivative of CD30.

The term “purified,” as used in the context of this specification to define the purity of CD30 protein or protein compositions, means that the protein or protein composition is substantially free of other proteins of natural or endogenous origin and contains less than about 1% by mass of protein contaminants residual of production processes. However, such compositions can contain other proteins added as stabilizers, carders, excipients or co-therapeutics.

The term “recombinant,” as used herein, means that a protein is derived from recombinant (e.g., mammalian) expression systems. Protein expressed in insect cells may have a glycosylation pattern different from that expressed in mammalian cells.

The term “therapeutically effective amount,” as used herein, means that an amount of CD30 or a biologically functional equivalent thereof is sufficient for an effective treatment when administered to a human in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “pharmaceutically acceptable,” as used herein, means that the carrier, diluent or excipient employed in the pharmaceutical composition according to the present invention must be compatible with the other ingredients of the composition and not deleterious to the recipient of the composition.

The term “locally,” as used herein, means the CD30 or the biologically functional equivalent thereof is injected into a confined space in a human body, for example, a joint space, the pleural space, or an eye ball.

CD30 Protein

The CD30 protein is not limited to a native CD30 protein but includes an artificial CD30 construct (e.g. genetically engineered CD30 construct). The native CD30 protein is a membrane protein. The mature full-length human CD30 is a glycoprotein having a molecular weight of about 110-120 kilodaltons (kDa). The CD30 protein employed according to the present invention may have an amino acid sequence corresponding to all or part of the extracellular region of a native CD30 and are biologically active. Preferably, the CD30 protein is capable of binding to a CD30 ligand molecule. In a preferred embodiment of the present invention, the CD30 protein is in soluble form.

Induction of CD30 ligand signal transduction activity can be determined by transfecting cells with recombinant CD30 ligand DNAs to obtain recombinant CD30 ligand expression. The transfected cells are then placed in contact with the CD30 protein, and the resulting metabolic effects examined. If an effect results which is attributable to the action of the CD30, then the recombinant CD30 ligand has signal transduction activity. Exemplary procedures for determining whether a polypeptide has signal transduction activity are disclosed by Idzerda et al., J. Exp. Med., 171:861 (1990); Curtis et al., Proc. Natl. Acad. Sci. U.S.A., 86:3045 (1989); Prywes et al., EMBO J., 5:2179 (1986) and Chou et al., J. Biol. Chem., 262:1842 (1987). Alternatively, primary cells or cell lines that express an endogenous CD30 ligand and have a detectable biological response to CD30 could also be utilized.

Biologically Functional Equivalent of CD30

The biologically functional equivalent of CD30 employed according to the present invention may have an amino acid sequence corresponding to all or part of the extracellular region of a native CD30 and are biologically active. In a preferred embodiment of the present invention, the biologically functional equivalent of CD30 is in soluble form. Preferably, the biologically functional equivalent may be an analog, a subunit, a mutant, an agonist polypeptide, a chimeric protein or a derivative of CD30.

Analog or Subunit of CD30

A soluble biologically functional equivalent of CD30 employed according to the present invention may include, for example, analogs or subunits of a native CD30 protein having at least 20 amino acids and exhibit at least some biological activity in common with CD30. The soluble biologically functional equivalent of CD30 may be devoid of a transmembrane region and intracellular domain but retain the ability to bind a CD30 ligand. The soluble biologically functional equivalents of CD30 protein may include polypeptides, varying from the amino acid sequence of native CD30 by one or more substitutions, deletions or additions and retain the ability to bind to a CD30 ligand and induce signal transduction activity via cell surface bound CD30 ligand proteins.

Induction of CD30 ligand signal transduction activity can be determined by transfecting cells with recombinant CD30 ligand DNAs to obtain recombinant CD30 ligand expression. The transfected cells are then placed in contact with the biologically functional equivalent of CD30, and the resulting metabolic effects examined. If an effect results which is attributable to the action of the biologically functional equivalent of CD30, then the recombinant CD30 ligand has signal transduction activity. Exemplary procedures for determining whether a polypeptide has signal transduction activity are disclosed by Idzerda et al., J. Exp. Med., 171:861 (1990); Curtis et al., Proc. Natl. Acad. Sci. U.S.A., 86:3045 (1989); Prywes et al., EMBO J., 5:2179 (1986) and Chou et al., J. Biol. Chem., 262:1842 (1987). Alternatively, primary cells or cell lines that express an endogenous CD30 ligand and have a detectable biological response to the biologically functional equivalent of CD30 could also be utilized.

Agonist Polypeptide of CD30

A biologically functional equivalent of CD30 employed according to the present invention may include purified CD30 agonist polypeptides. The purified CD30 agonist polypeptides used in this invention are substantially free of other contaminating materials of natural or endogenous origin and contain less than about 1% by mass of protein contaminants residual of production processes. The CD30 agonist polypeptides used in this invention are optionally modified with glycosylation.

Like most other mammalian proteins, the CD30 protein is encoded by a multi-exon gene. Alternative mRNA constructs that can be attributed to different mRNA splicing events following transcription and share large regions of identity or similarity with the cDNA first published (Durkop et al., Cell, 68:421, 1992) may also be used.

Other mammalian CD30 cDNAs may be isolated by using appropriate human CD30 DNA sequences as a probe for screening a particular mammalian cDNA library by cross-species hybridization. Mammalian CD30 cDNAs can be obtained by cross species hybridization, using a single stranded cDNA derived from the human CD30 DNA sequence as a hybridization probe to isolate CD30 cDNAs from mammalian cDNA libraries.

Derivative of CD30

A biologically functional equivalent of CD30 employed according to the present invention may include derivatives of CD30. The derivatives of CD30 may also include various structural forms of the primary protein, which retain biological activity. Due to the presence of ionizable amino and carboxyl groups, for example, a CD30 protein may be in the form of acidic or basic salts, or may be in neutral form. Individual amino acid may be modified by oxidation or reduction.

The primary structure of the CD30 protein according to the present invention may be modified by forming covalent or aggregative conjugates with other chemical moieties, such as glycosyl groups, lipids, phosphate, acetyl groups and the like, or by creating amino acid sequence mutants. Covalent derivatives are prepared by linking particular functional groups to CD30 amino acid side chains or at the N- or C-termini. Other derivatives of CD30 include covalent or aggregative conjugates of CD30 or its fragments with other proteins or polypeptides, such as by synthesis in recombinant culture as N-terminal or C-terminal fusions. For example, the conjugated peptide may be a signal (or leader) polypeptide sequence at the N-terminal region of the protein. The signal peptide co-translationally or post-translationally directs transfer of the protein from its site of synthesis to its site of function inside or outside of the cell membrane or wall (e.g., the yeast alpha-factor leader). The CD30 protein fusions can comprise peptides added to facilitate purification or identification of CD30 (e.g., poly-His or Fc domain of immunoglobulin molecule). The amino acid sequence of CD30 can also be linked to the peptide Asp-Tyr-Lys-Asp-Asp-Asp-Asp-Lys (DYKDDDDK) (Hopp et al., Bio/Technology., 6:1204, 1988.) The latter sequence is highly antigenic and provides an epitope reversibly bound by a specific monoclonal antibody, enabling rapid assay and facile purification of expressed recombinant protein. This sequence is also specifically cleaved by bovine mucosal enterokinase at the residue immediately following the Asp-Lys pairing.

CD30 with or without associated native-pattern glycosylation may also be used. CD30 expressed in yeast or mammalian expression systems, e.g., COS-7 cells, may be similar or slightly different in molecular weight and glycosylation pattern than the native molecules, depending upon the expression system. Expression of CD30 DNAs in bacteria such as E. coli provides non-glycosylated molecules.

Mutant of CD30

A biologically functional equivalent of CD30 employed according to the present invention may include mutants of CD30. A CD30 mutant, as referred to herein, is a polypeptide homologous to CD30 but which has an amino acid sequence different from native CD30 because of a deletion, insertion or substitution.

The CD30 mutant may be constructed by, for example, making various substitutions of residues or sequences or deleting terminal or internal residues or sequences not needed for biological activity. For example, cysteine residues can be deleted or replaced with other amino acids to prevent formation of unnecessary or incorrect intramolecular disulfide bridges upon renaturation. Other approaches to mutagenesis involve modification of adjacent dibasic amino acid residues to enhance expression in yeast systems in which KEX2 protease activity is present. Generally, substitutions should be made conservatively; i.e., the most preferred substitute amino acids are those having physiochemical characteristics resembling those of the residue to be replaced. Similarly, when a deletion or insertion strategy is adopted, the potential effect of the deletion or insertion on biological activity should be considered. Substantially similar polypeptide sequences, as defined above, generally comprise a like number of amino acid sequences. To preserve the biological activity of CD30 proteins, deletions and substitutions will preferably result in homologous or conservatively substituted sequences, meaning that a given amino acid residue is replaced by a biologically similar residue. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Moreover, particular amino acid differences between human, murine and other mammalian CD30 is suggestive of additional conservative substitutions that may be made without altering the essential biological characteristics of CD30.

The CD30 mutant may be constructed by deleting terminal or internal residues or sequences. Particularly preferred sequences include those in which the transmembrane region and intracellular domain of CD30 are deleted or substituted with hydrophilic residues to facilitate secretion of the receptor into the cell culture medium. The resulting protein is referred to as a soluble CD30 mutant that retains its ability to bind to CD30 ligand. A particularly preferred soluble CD30 mutant construct is one that comprises the entire extracellular region of CD30 terminating with Lys immediately adjacent to the transmembrane region. Additional amino acids may be deleted from the extracellular region of CD30, while retaining CD30 ligand binding activity. The present invention contemplates use of such soluble CD30 mutant constructs corresponding to all or part of the extracellular region of CD30. Other C-terminal deletions may be made as a matter of convenience by cutting CD30 cDNA with appropriate restriction enzymes and, if necessary, reconstructing specific sequences with synthetic oligonucleotide linkers. Soluble CD30 mutant with N-terminal deletions may also be used in the present invention. The resulting soluble CD30 mutant constructs are then inserted and expressed in appropriate expression vectors and assayed for the ability to bind to CD30 ligands.

Mutations in nucleotide sequences which are constructed for expression of the biologically functional equivalent of CD30, must preserve the reading frame phase of the coding sequences and preferably will not create complementary regions that could hybridize to produce secondary mRNA structures such as loops or hairpins that would adversely affect translation of the protein mRNA. Although a mutation site may be predetermined, predetermining the nature of the mutation per se is not necessary. For example, to select optimum characteristics of mutants at a given site, random mutagenesis may be conducted at the target codon and the expressed CD30 mutants screened for the desired activity.

Not all mutations in the nucleotide sequence encoding a biologically functional equivalent of CD30 will be expressed in the final product. For example, nucleotide substitutions may be made to enhance expression, primarily to avoid secondary structure loops in the transcribed mRNA or to provide codons that are more readily translated by the selected host, e.g., the well-known E. coli preference codons for E. coli expression.

Mutations can be introduced at particular loci flanked by restriction sites enabling ligation to fragments of the native sequence by synthesizing oligonucleotides containing a mutant sequence. Following ligation, the resulting reconstructed sequence encodes a biologically functional equivalent of CD30 having the desired amino acid insertion, substitution or deletion.

Alternatively, oligonucleotide-directed site-specific mutagenesis procedures can be employed to provide an altered gene having particular codons altered according to the substitution, deletion or insertion required. Exemplary methods of making the alterations set forth above are disclosed by Walder et al. (Gene, 42:133, 1986); Bauer et al. (Gene, 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462.

Chimeric Protein

A biologically functional equivalent of CD30 employed according to the present invention may include a recombinant chimeric antibody molecule having a part of CD30 sequences substituted for the variable domains of either or both of the immunoglobulin molecule heavy and light chains and having unmodified constant region domains. For example, chimeric CD30/IgG may be produced from two chimeric genes; a CD30/human kappa light chain chimera and a CD30/human gamma heavy chain chimera. Following transcription and translation of the two chimeric genes, the gene products assemble into a single chimeric antibody molecule having CD30 displayed tetravalently. The gene for CD30/human gamma heavy chain chimera alone may be used to produce a single chimeric antibody molecule having CD30 displayed bivalently. Such polyvalent forms of CD30 may have enhanced binding affinity for CD30 ligand. Additional details relating to the construction of such chimeric antibody molecules are disclosed in WO 89/09622 and EP 315062.

Monovalent Form and Polyvalent Form of CD30 or a Biologically Functional Equivalent of CD30

Both monovalent forms and polyvalent forms of CD30 or a biologically functional equivalent of CD30 may also be used in the present invention. Polyvalent forms possess multiple CD30 ligand binding sites. For example, a bivalent soluble CD30 may consist of two tandem repeats of amino acids corresponding to the extracellular domain of CD30, separated by a linker region. Alternate polyvalent forms may also be constructed, for example, by chemically coupling CD30 or the biologically functional equivalent of CD30 to any clinically acceptable carder molecule, a polymer selected from the group consisting of Ficoll, polyethylene glycol or dextran using conventional coupling techniques. Alternatively, CD30 or the biologically functional equivalent of CD30 may be chemically coupled to biotin, and the biotin-CD30 or biotin-biologically functional equivalent of CD30 conjugate then allowed to bind to avidin, resulting in tetravalent avidin/biotin/CD30 or biologically functional equivalent of CD30 molecules. CD30 or the biologically functional equivalent of CD30 may also be covalently coupled to dinitrophenol (DNP) or trinitrophenol (TNP) and the resulting conjugate precipitated with anti-DNP or anti-TNP-IgM, to form decameric conjugates with a valency of 10 for CD30 ligand binding sites.

Expression of CD30

Recombinant expression vectors are preferably used to amplify or express DNA encoding CD30 or the biologically functional equivalent of CD30 to obtain purified CD30 or the biologically functional equivalent of CD30. Recombinant expression vectors are replicable DNA constructs which have synthetic or cDNA-derived DNA fragments encoding mammalian CD30 or the biologically functional equivalent of CD30 operably linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence transcribed into mRNA and translated into protein, and (3) appropriate transcription and translation initiation and termination sequences, as described in detail below. Such regulatory elements may include an operator sequence to control transcription, a sequence encoding suitable mRNA ribosomal binding sites. The ability to replicate in a host, usually conferred by an origin of replication and a selection gene to facilitate recognition of transformants may additionally be incorporated. DNA regions are operably linked when they are functionally related to each other. For example, DNA for a signal peptide (secretory leader) is operably linked to DNA for a polypeptide if it is expressed as a precursor that participates in the secretion of the polypeptide. A promoter is operably linked to a coding sequence if it controls the transcription of the sequence. A ribosome-binding site is operably linked to a coding sequence if it is positioned to permit translation. Generally, “operably linked” means contiguous and, in the case of secretory leaders, in a reading frame. Structural elements intended for use in yeast expression systems preferably include a leader sequence enabling extracellular secretion of translated protein by a host cell. Alternatively, where recombinant protein is expressed without a leader or transport sequence, the recombinant protein may include an N-terminal methionine residue. This residue may optionally be subsequently cleaved from the expressed recombinant protein to provide a final product.

DNA sequences encoding mammalian CD30 proteins that are to be expressed in a cell will preferably contain no introns that could prematurely terminate transcription of DNA into mRNA. However, premature termination of transcription may be desirable, for example, where it would result in mutants having advantageous C-terminal truncations, for example, deletion of a transmembrane region to yield a soluble receptor not bound to the cell membrane. Due to code degeneracy, there can be considerable variation in nucleotide sequences encoding the same amino acid sequence. Other embodiments include sequences capable of hybridizing to the sequences of the provided cDNA under moderately stringent conditions and other sequences hybridizing or degenerate to those which encode biologically active CD30 polypeptides.

Recombinant CD30 DNA is expressed or amplified in a recombinant expression system, comprising a substantially homogeneous monoculture of suitable host microorganisms or cells, that have stably integrated (by transformation or transfection) a recombinant transcriptional unit into chromosomal DNA or carry the recombinant transcriptional unit as a component of a resident plasmid. Generally, cells constituting the system are the progeny of a single ancestral transformant. Recombinant expression systems as defined herein will express heterologous protein upon induction of the regulatory elements linked to the DNA sequence or synthetic gene to be expressed.

Transformed host cells have been transformed or transfected with CD30 vectors constructed using recombinant DNA techniques. Transformed host cells ordinarily express CD30, but host cells transformed for purposes of cloning or amplifying CD30 DNA do not need to express CD30. Expressed CD30 will be deposited in the cell membrane or secreted into the culture supernatant, depending on the CD30 DNA selected. Suitable host cells for expression of mammalian CD30 include prokaryotes, yeast, insect cells or higher eukaryotic cells under the control of appropriate promoters. Prokaryotes include gram negative or gram positive organisms, for example E. coli or bacilli. Higher eukaryotic cells include established cell lines of mammalian origin as described below. Cell-free translation systems could also be employed to produce mammalian CD30 using RNAs derived from the DNA constructs of the present invention. Appropriate cloning and expression vectors for use with bacterial, fungal, yeast, and mammalian cellular hosts are described by Pouwels et al. (Cloning Vectors: A Laboratory Manual, Elsevier, N.Y., 1985).

Prokaryotic expression hosts may be used for expression of CD30 that do not require extensive proteolytic and disulfide processing. Prokaryotic expression vectors generally comprise one or more phenotypic selectable markers, for example a gene encoding proteins conferring antibiotic resistance or supplying an autotrophic requirement and an origin of replication recognized by the host to ensure amplification within the host. Suitable prokaryotic hosts for transformation include E. coli, Bacillus subtilis, Salmonella typhimurium, and various species within the genera Pseudomonas, Streptomyces, and Staphyolococcus, although others may also be employed as a matter of choice.

Useful expression vectors for bacterial use can comprise a selectable marker and bacterial origin of replication derived from commercially available plasmids comprising genetic elements of the well-known cloning vector pBR322 (ATCC 37017). Such commercial vectors include, for example, pKK223-3 (Pharmacia Fine Chemicals, Uppsala, Sweden) and pGEM1 (Promega Biotec, Madison, Wis. U.S.A.). These pBR322 “backbone” sections are combined with an appropriate promoter and the structural sequence to be expressed. E. coli is typically transformed using derivatives of pBR322, a plasmid derived from an E. coli species (Bolivar et al., Gene, 2:95, 1977). pBR322 contains genes for ampicillin and tetracycline resistance and thus provides simple means for identifying transformed cells.

Promoters commonly used in recombinant microbial expression vectors include the beta-lactamase (penicillinase) and lactose promoter system (Chang et al., Nature, 275:615, 1978; and Goeddel et al., Nature, 281:544, 1979), the tryptophan (trp) promoter system (Goeddel et al., Nucl. Acids Res., 8:4057, 1980; and EPA, 36, 776) and tac promoter (Maniatis, Molecular Cloning: A Laboratory Manual, Cold Spring Harbor Laboratory, p. 412, 1982).

Recombinant CD30 proteins may also be expressed in yeast hosts, preferably from the Saccharomyces species, such as S. cerevisiae. Yeast of other genera, such as Pichia or Kluyveromyces may also be employed.

Various mammalian or insect cell culture systems are also advantageously employed to express recombinant protein. Expression of recombinant proteins in mammalian cells is particularly preferred because such proteins are generally correctly folded, appropriately modified and completely functional. Examples of suitable mammalian host cell lines include the COS-7 lines of monkey kidney cells, described by Gluzman (Cell, 23:175, 1981), and other cell lines capable of expressing an appropriate vector including, for example, L cells, C127, 3T3, Chinese hamster ovary (CHO), HeLa and BHK cell lines. Mammalian expression vectors may comprise nontranscribed elements such as an origin of replication, a suitable promoter and a suitable enhancer linked to the gene to be expressed, and other 5′ or 3′ flanking nontranscribed sequences, and 5′ or 3′ nontranslated sequences, such as necessary ribosome-binding sites, a polyadenylation site, splice donor and acceptor sites, and transcriptional termination sequences. Baculovirus systems for production of heterologous proteins in insect cells are reviewed by Luckow and Summers, Bio/Technology, 6:47 (1988).

The transcriptional and translational control sequences in expression vectors to be used in transforming vertebrate cells may be provided by viral sources. For example, commonly used promoters and enhancers are derived from Polyoma, Adenovirus 2, Simian Virus 40 (SV40) and human cytomegalovirus. DNA sequences derived from the SV40 viral genome, for example, SV40 origin, early and late promoter, enhancer, splice and polyadenylation sites may be used to provide the other genetic elements required for expression of a heterologous DNA sequence. The early and late promoters are particularly useful because both are obtained easily from the virus as a fragment that also contains the SV40 viral origin of replication (Fiers et al., Nature, 273:113, 1978). Smaller or larger SV40 fragments may also be used, provided the approximately 250 bp sequence extending from the HindIII site toward the BglI site located in the vital origin of replication is included. Further, mammalian genomic CD30 promoter, control and/or signal sequences may be utilized, provided such control sequences are compatible with the host cell chosen. Additional details regarding the use of a mammalian high expression vector to produce a recombinant mammalian CD30 are provided in examples below. A useful system for stably high level expression of mammalian receptor cDNAs in C127 murine mammary epithelial cells can be constructed substantially as described by Cosman et al. (Mol. Immunol. 23:935, 1986).

Recombinant expression vectors comprising CD30 cDNAs are stably integrated into a host cell's DNA. Elevated levels of expression product are achieved by selecting cell lines having amplified numbers of vector DNA. Cell lines having amplified numbers of vector DNA are selected, for example, by transforming a host cell with a vector comprising a DNA sequence encoding an enzyme that is inhibited by a known drug. The vector may also comprise a DNA sequence encoding a desired protein. Alternatively, the host cell may be co-transformed with a second vector comprising the DNA sequence encoding the desired protein. The transformed or co-transformed host cells are then cultured in increasing concentrations of the known drug, thereby selecting drug-resistant cells. Such drug-resistant cells survive in increased concentrations of the toxic drug by over-production of the enzyme that is inhibited by the drug, frequently as a result of amplification of the gene encoding the enzyme. Where drug resistance is caused by an increase in the copy number of the vector DNA encoding the inhibitable enzyme, there is a concomitant co-amplification of the vector DNA encoding the desired protein in the host cell's DNA.

A preferred system for such co-amplification uses the gene for dihydrofolate reductase (DHFR), which can be inhibited by the drug methotrexate (MTX). To achieve co-amplification, a host cell that lacks an active gene encoding DHFR is either transformed with a vector that comprises DNA sequence encoding DHFR and a desired protein or is co-transformed with a vector comprising a DNA sequence encoding DHFR and a vector comprising a DNA sequence encoding the desired protein. The transformed or co-transformed host cells are cultured in media containing increasing levels of MTX, and those cells lines that survive are selected.

A particularly preferred co-amplification system uses the gene for glutamine synthetase (GS), which is responsible for the synthesis of glutamate and ammonia using the hydrolysis of ATP to ADP and phosphate to drive the reaction. GS is subject to inhibition by a variety of inhibitors, for example methionine sulphoximine (MSX). Thus, CD30 can be expressed in high concentrations by cells transformed with a vector comprising the DNA sequence for GS and CD30, or co-transformed with a vector comprising a DNA sequence encoding GS and a vector comprising a DNA sequence encoding CD30. These cells are selected by culturing the host cells in media containing increasing levels of MSX and selecting surviving cells. The GS co-amplification system, appropriate recombinant expression vectors and cells lines are described in the PCT applications WO 87/04462, WO 89/01036, WO 89/10404 and WO 86/05807.

A preferred eukaryotic vector for expression of CD30 DNA is disclosed in the examples that follow. This vector, referred to as pMIB, contains regulatory sequences from honeybee.

Purification of CD30

Purified mammalian CD30 or the biological functional equivalent are prepared by culturing suitable host/vector systems to express the recombinant translation products, which are then purified from culture media or cell extracts.

For example, supernatants from systems that secrete recombinant protein into culture media can be first concentrated using a commercially available protein concentration filter, for example, an Amicon or Millipore ultrafiltration unit. Following the concentration step, the concentrate can be applied to a suitable purification matrix. A suitable affinity matrix can comprise an antibody molecule bound to a suitable support. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred.

Finally, one or more reversed-phase high performance liquid chromatography (RP-HPLC) steps employing hydrophobic RP-HPLC media, e.g., silica gel having pendant methyl or other aliphatic groups, can be employed to further purify a CD30 composition. Some or all of the foregoing purification steps, in various combinations, can also be employed to provide a homogeneous recombinant protein.

Recombinant protein produced in bacterial culture is usually isolated by initial extraction from cell pellets, followed by one or more concentration, salting-out, aqueous ion exchange or size exclusion chromatography steps. Finally, high performance liquid chromatography (HPLC) can be employed for final purification steps. Microbial cells employed in expression of recombinant mammalian CD30 can be disrupted by any convenient method, including freeze-thaw cycling, sonication, mechanical disruption or use of cell lysing agents.

Fermentation of yeast that expresses mammalian CD30 as a secreted protein greatly simplifies purification. Secreted recombinant protein resulting from large-scale fermentation can be purified by methods analogous to those disclosed by Urdal et al. (J. Chromatog., 296:171, 1984). This reference describes two sequential, reversed-phase HPLC steps for purification of recombinant human GM-CSF on a preparative HPLC column.

Human CD30 synthesized in recombinant culture is characterized by the presence of non-human cell components, including proteins, in amounts and of a character that depend upon the purification steps taken to recover human CD30 from the culture. These components ordinarily will be of yeast, prokaryotic or non-human higher eukaryotic origin and preferably are present in innocuous contaminant quantities, on the order of less than about 1 percent by weight. Further, recombinant cell culture enables the production of CD30 free of proteins which may be normally associated with CD30 as it is found in nature in its species of origin.

The Effect of Soluble CD30 or Plate-bound CD30 on T Cells

The present invention provides a method of lowering the levels of T cell proliferation and activation in activated human T cells, comprising administering an effective amount of CD30 or a biologically functional equivalent thereof. Preferably, the biologically functional equivalent thereof is a chimeric protein of CD30, such as CD30-Fc. More preferably, the biologically functional equivalent is a chimeric antibody comprising an extracellular domain of CD30 fused to an immunoglobulin heavy chain constant region polypeptide. Anti-CD3 treatment of T cells is considered an in-vitro method to mimic T cell activation in vivo. Tritium thymidine incorporation is the method employed in the present invention to measure the proliferation responses of activated T cells. The amount of IL-2 secreted by activated T cells into the medium was also measured. IL-2 is the key cytokine responsible for the proliferative responses after T cell activation, and can play in either an autocrine or a paracrine manner. The CD30 or the biologically functional equivalent thereof is able to decrease the production of IL-2 from anti-CD3-treated T cells. To measure the levels of T cell activation, the amount of CD25 and CD26 expression in activated T cells was measured. Both of these surface markers are known to express after adequate T cell activation in vitro or in vivo. The decrease in the levels of T-cell proliferation by a therapeutically effective amount of CD30 or a biologically functional equivalent thereof can be attenuated with IL-2 supplementation.

In summary, the present invention established that CD30 is involved in the inhibition of T-cell proliferation, IL-2 production, and expression of CD25 and CD26 by T cells. Controlling T-cell proliferation or T-cell activation could be a method for treating immune disorders.

The present invention provides a method for treating immune disorders comprising administering CD30 or a biologically functional equivalent thereof to a human afflicted with immune disorders. The CD30 or the biologically functional equivalent thereof can be administered intravenously and be administered in the form of a composition that additionally comprises a diluent, excipient or carrier. The biologically functional equivalent is a soluble chimeric protein. The immune disorders are associated with T-cell activation, T-cell proliferation, T-cell production of IL-2, T-cell expression of CD25 or CD26.

The present invention also provides a pharmaceutical composition for treating immune disorders in a human comprising a therapeutically effective amount of CD30 or a biologically functional equivalent thereof, and a pharmaceutically acceptable carrier, excipient or diluent.

The pharmaceutical composition is prepared by known procedures using well-known and readily available ingredients. The compositions of this invention may be formulated to provide quick, sustained or delayed release of the active ingredient after administration to the patient by employing procedures well-known in the art. In making the compositions of the present invention, the active ingredient will usually be admixed with a carrier, diluent or excipient. The carrier may be in the form of a capsule, sachet, paper or other container. The diluent may be a solid, semi-solid or liquid material that acts as a vehicle, excipient or medium for the active ingredient. The excipient may be starch, sucrose, starch, glycol, mannitol, microcrystalline cellulose and so on.

All of the documents or publications recited in the text are incorporated herein by reference.

The following examples are offered by way of illustration, and not by way of limitation.

### EXAMPLES

### Example 1

Construction and Expression of Soluble Human CD30-Fc Fusion Protein

The cloning of the cDNA for the human CD30 has been described in detail (Durkop et al., Cell, 68:421, 1992).

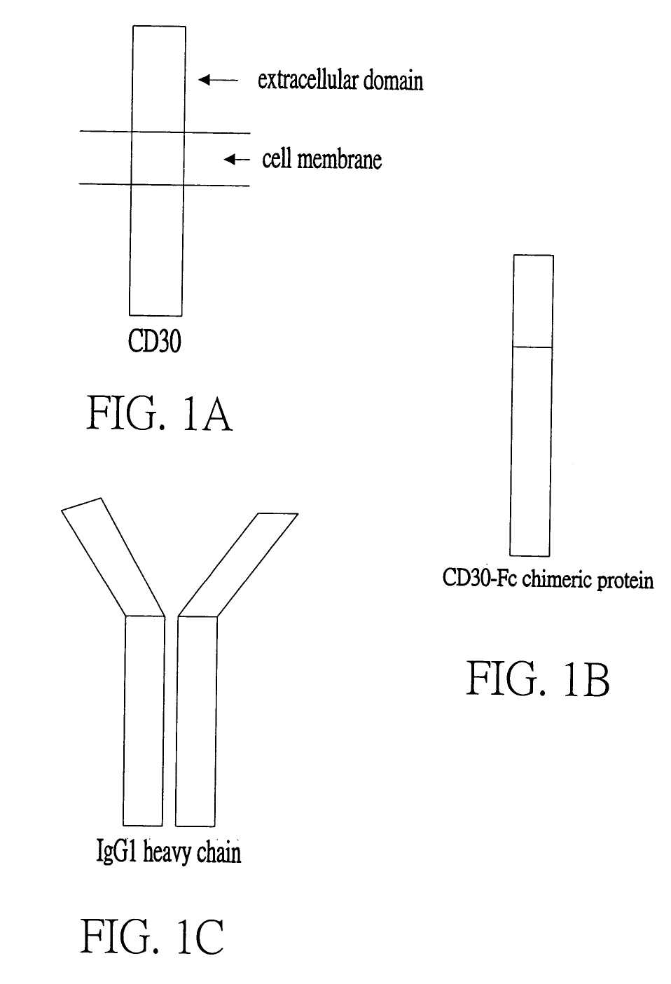

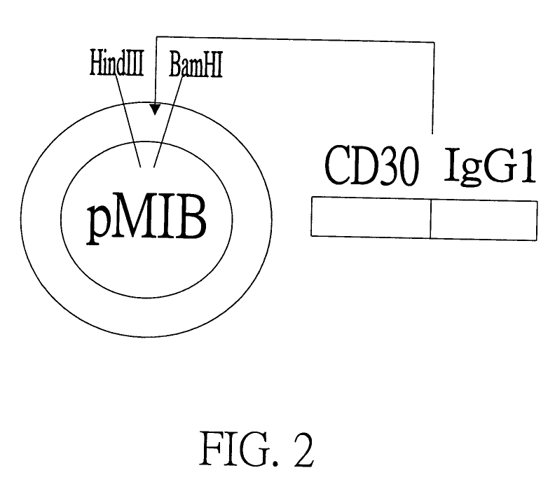

With reference to FIGS. 1 and 2, the gene segment for the CD30-Fc fusion protein was transferred to the MIB vector (Invitrogen, San Diego, Calif.). The primary translational product of the plasmid coding for rhu (recombinant human) CD30-Fc is a single molecule of soluble CD30 (corresponding to the extracellular domain of the protein) linked to a single chain of Fc derived from human IgG1. Following translation, this fusion molecule dimerizes via cysteine residues in the Fc region to form dimeric rhu CD30-Fc. The gene segment for rhu CD30-Fc is inserted into pMIB, a commercially available plasmid for expression of a desired protein in insect cells.

The rhu CD30-Fc fusion protein was created by ligating the following fragments (a) and (b).

(a) The first segment contains the protein segment for the extracellular domain of CD30. The corresponding gene segment was generated by PCR by use of the sense oligonucleotides 5′-AAGAATTCTTCCCACAGGATCGAC and the anti-sense oligonucleotides 5′-TTGTTAACCTTCCCCGTGGAGGAGAG.

(b) The other protein segment contains a 238 amino acid polypeptide, derived from a 715 bp fragment from human IgG1, coding for the Fc portion of human IgG1.

The pMIB containing the gene for rhu CD30-Fc was transfected into the insect cells Sf21. High producers were selected with ELISA kits for the human immunoglobulin Fc portion. Purification was done with protein A columns (Amersham Pharmacia, Uppsala, Sweden). Bound antibodies were eluted with a buffer containing 0.1 mol/L glycine 0.15 mol/L NaCl, pH 2.4 and brought to neutral pH with 0.5 mol/L sodium phosphate, pH 8.0. Purified proteins were dialyzed extensively with PBS (phosphate buffered saline) and sterilized by filtration. The concentrations of proteins were determined by a bicinchoninic acid-based protein assay (Pierce, Rockford, Ill.).

Transfections were performed by mixing pMIB/rhuCD30-Fc plasmid DNA with Lipofectin from Gibco BRL. Approximately 10 μg of DNA was added to 10 cm petri dishes containing Sf21 cells. After the initial transfection, cells were selected in a selective medium containing 50 μg/ml Blasticidin. The resulting colonies were then transferred to 24 well plates and analyzed for rhu CD30-Fc expression. The highest expressing cultures were subjected to amplification by exposure to increasing concentrations of Blasticidin. Cells able to grow were cloned by limiting dilution in 96 well plates. The highest expressing clones were transferred to a suspension culture, and the final selection of clones were made based on its high level of rhu CD30-Fc expression under these conditions.

### Example 2

Effect of CD30-Fc on T Cell Proliferation

Total peripheral blood mononuclear cells (PBMCs) were separated on Ficoll-Hypaque (Amersham Biosciences, Uppsala, Sweden). CD3+ and CD4+ T cells in PBMCs were affinity-purified by use of the CD3+ and CD4+ T MACS isolation kits respectively (Miltenyi Biotech, Bergisch Gladbach, Germany) with a negative selection method. The resulting purified cells were more than 95% positive for CD3 or CD4. The retained cells in the column were eluted outside the magnetic field, treated with 2% paraformaldehyde (J.T. Baker, Phillipsburg, N.J.) for two hours, and washed extensively with PBS. These cells were used as accessory cells in the T-cell proliferation assay.

To study the effect of the CD30-Fc fusion protein on T-cell proliferation, plate-bound and soluble CD30-Fc fusion protein was used. Plates with different concentrations of CD30-Fc chimeric protein or human IgG (Cappel, Aurora, Ohio) were stored at 4° C. overnight and washed with PBS before use. T cells and accessory cells (each 1×10⁵ cells/well) were treated with soluble anti-CD3 (HIT3a, BD PharMingen, San Diego, Calif.) at 0.5 μg/ml and different concentrations of CD30-Fc fusion protein or human IgG and were incubated in triplicate in U-bottom 96-well plates at 37° C./5% CO₂ for 3 days. During the last 6 hours of incubation, cultures were pulsed with 0.5 μCi/well [3H]thymidine. Cells were then harvested with a Packard Cell Harvester onto unifilter plates. Cell-associated radioactivity was measured by scintillation counting. In other studies, soluble CD30-Fc or IgG was substituted for plate-bound CD30-Fc or IgG.

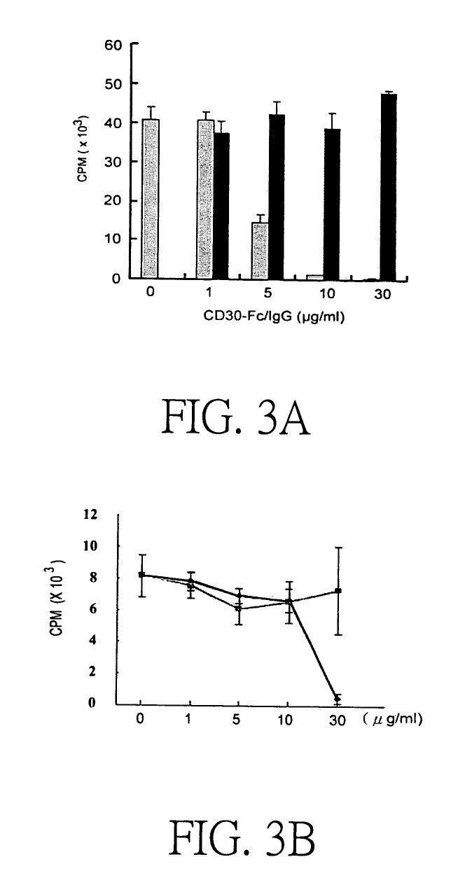

As shown in FIG. 3A, plate-bound CD30-Fc fusion protein had a dose-dependent inhibitory effect on the proliferation of anti-CD3-treated T cells. A dramatic inhibition was observed when the concentrations were equal to or more than 10 μg/ml. CD30 inhibits anti-CD3-induced T-cell proliferation, while human IgG had little effect. In FIG. 3B, a similar dose-dependent inhibitory effect on T cell proliferation was observed, although higher concentrations were required to achieve a similar effect on the inhibition of T cell proliferation, when soluble CD30-Fc was substituted for plate-bound CD30-Fc.

### Example 3

Effect of CD30-Fc on IL-2 Production from Activated T Cells.

To study the effect of CD30-Fc on IL-2 production from anti-CD3-treated T cells, IL-2 amounts in the culture supernatant were measured. T cells and accessory cells (1×10⁵ cells/well for each type of cells) were co-cultured in triplicate in U-bottom 96-well plates with soluble anti-CD3 at 0.5 μg/ml and different concentrations of the plate-bound CD30-Fc chimeric protein. Plates were incubated at 37° C./5% CO₂ for 3 days. The supernatant was collected, and levels of IL-2 production were measured with an IL-2 ELISA Kit (Diaclone, France) according to the manufacturer's protocol.

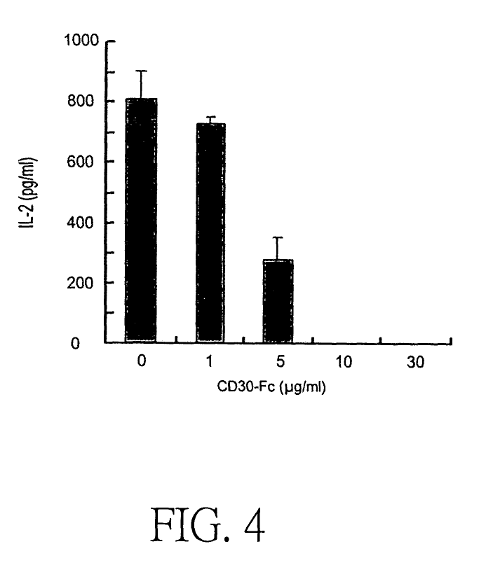

IL-2 production from anti-CD3-treated T cells co-cultured with higher concentrations of CD30-Fc chimeric protein was not detectable (FIG. 4). CD30-Fc is able to inhibit the production of IL-2 from anti-CD3-treated T cells.

### Example 4

Effect of IL-2 Addition on the Inhibition of T Cell Proliferation by CD30-Fc.

To study the effect of IL-2 addition on T-cell proliferation, recombinant human IL-2 (United States Biological, Swampscott, Mass.) at 1 ng/ml was used. T cells, accessory cells (1×10⁵ cells/well for each type of cells) and different concentrations of the plate-bound CD30-Fc chimeric protein were co-cultured in triplicate in U-bottom 96-well plates with soluble anti-CD3 at 0.5 μg/ml and IL-2. Plates were incubated at 37° C./5% CO₂ for 3 days. During the last 6 h of incubation, cultures were pulsed with 0.5 μCi/well of [3H]thymidine. Cells were then harvested with a Packard Cell Harvester onto unifilter plates. Cell-associated radioactivity was measured by scintillation counting.

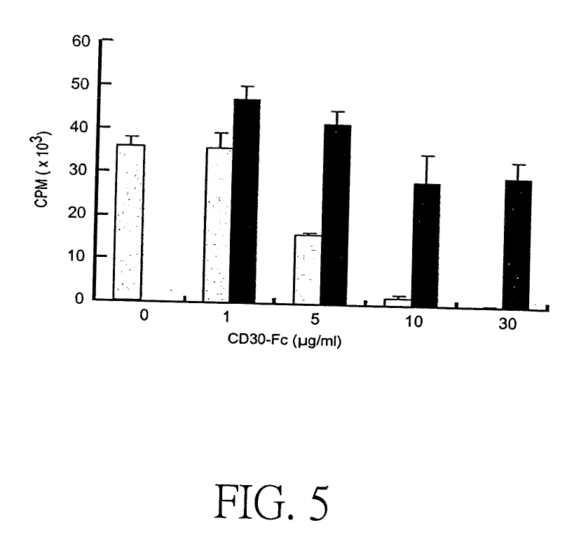

Addition of exogenous IL-2 restored T-cell proliferation inhibited by CD30-Fc fusion protein (FIG. 5), supporting the claim that the decrease in the levels of T-cell proliferation by CD30 is attenuated with IL-2 supplementation.

### Example 5

CD30-Fc Chimeric Protein Inhibit Expression of CD25 and CD26 by Anti-CD3-treated T Cells

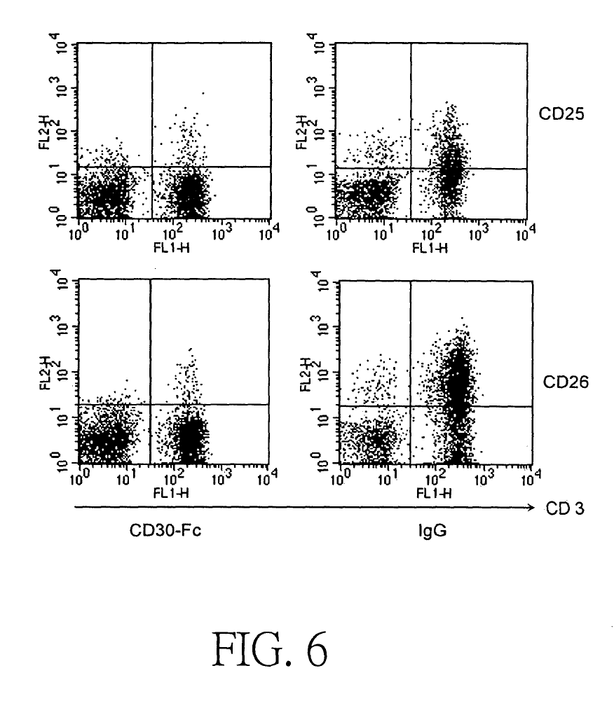

T cells in the co-cultures with different concentrations of the plate-bound CD30-Fc chimeric protein or human IgG were analyzed for the expression of CD25 or CD26 with FACS (BD Immunocytometry Systems, San Jose, Calif.). FITC-labeled CD3 and PE-labeled CD25/CD26 mouse monoclonal antibodies were used. For negative controls, FITC- and PE-labeled mouse IgGs were substituted. All antibodies were purchased from BD PharMingen. As shown in FIG. 6, CD30-Fc chimeric protein was able to inhibit the expression of CD25 and CD26 in anti-CD3-treated T cells. The X axis of the dot plots stand for CD3, and the Y-axis for CD25 or CD26.

Although the invention has been explained in relation to its preferred embodiments, many other possible modifications and variations can be made without departing from the spirit and scope of the invention as hereinafter claimed.

1. A method for treating immune disorders comprising administering CD30 or a biologically functional equivalent thereof to a human afflicted with immune disorders.

2. The method as claimed in claim 1, wherein the CD30 or the biologically functional equivalent thereof is administered intravenously.

3. The method as claimed in claim 1, wherein the CD30 or the biologically functional equivalent thereof is administered locally.

4. The method as claimed in claim 1, wherein the CD30 or the biologically functional equivalent thereof is a soluble protein.

5. The method as claimed in claim 1, wherein the biologically functional equivalent is a soluble chimeric protein.

6. The method as claimed in claim 1, wherein the CD30 or the biologically functional equivalent thereof is administered in the form of a composition that additionally comprises a diluent, excipient or carrier.

7. The method as claimed in claim 1, wherein the immune disorders are associated with T-cell activation.

8. The method as claimed in claim 1, wherein the immune disorders are associated with T-cell proliferation.

9. A method for lowering the levels of T-cell activation and T-cell proliferation in a human in need of lowering the levels of T-cell activation and T-cell proliferation, which comprises administering to a human a therapeutically effective amount of CD30 or a biologically functional equivalent thereof.

10. The method as claimed in claim 9, wherein the biologically functional equivalent is a chimeric antibody comprising an extracellular domain of CD30 fused to an immunoglobulin heavy chain constant region polypeptide.

11. The method as claimed in claim 9, wherein the T cell activation is associated with T-cell production of IL-2.

12. The method as claimed in claim 9, wherein the T cell activation is associated with T-cell expression of CD25.

13. The method as claimed in claim 9, wherein the T cell activation is associated with T-cell expression of CD26.

14. The method as claimed in claim 9, wherein lowering the level of T cell activation is associated with the decrease in the level of IL-2 production.

15. The method as claimed in claim 9, wherein the decrease in the level of T-cell proliferation by a therapeutically effective amount of CD30 or a biologically functional equivalent thereof is attenuated with IL-2 supplementation.

16. A pharmaceutical composition for treating immune disorders in a human comprising a therapeutically effective amount of CD30 or a biologically functional equivalent thereof and a pharmaceutically acceptable carrier, excipient or diluent.

17. The pharmaceutical composition as claimed in claim 16, wherein the CD30 or the biologically functional equivalent thereof is a soluble protein.

18. The pharmaceutical composition as claimed in claim 16, wherein the biologically functional equivalent is a soluble chimeric protein.

19. The pharmaceutical composition as claimed in claim 16, wherein the CD30 or the biologically functional equivalent binds to CD30 ligand.
